(19) United States
(12) Patent Application Publication
(10) Pub. No.: US 2005/0048540 A1
(43) Pub. Date: Mar. 3, 2005

(54) Chip for processing of gene and apparatus for processing of gene

(76) Inventors: Hisao Inami, Matsudo (JP); Yasuhiko Sasaki, Tsuchiura (JP); Ryo Miyake, Tsukuba (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/870,064

(22) Filed: Jun. 18, 2004

(30) Foreign Application Priority Data
Aug. 26, 2003 (JP) 2003-300696

(51) Int. Cl. C12Q 1/68; C12M 1/34

(52) U.S. Cl. 435/6; 435/287.2

(57) ABSTRACT

The present invention provides an analytical chip that is easy to handle, inexpensive, and for which the extraction of gene from a sample and analysis thereof can be automated to one process, and a small-sized and portable analytical apparatus equipped therewith. The chip for processing of gene that is equipped with an injection port into which a sample containing gene is delivered, a gene extraction part into which a solution containing said sample is introduced and which has a gene-binding carrier that binds to said gene, a washing solution-storing part that stores the washing solution to be introduced into said gene extraction part, and a reaction part into which said gene captured in said extraction part is introduced, wherein a fluid channel through which said washing solution is introduced from said washing solution-storing part has been connected to a region more remote from said injection port than from a region into which a solution containing said sample is introduced in said gene extraction part is obtained.

### INCORPORATION BY REFERENCE

The present application claims priority of Japanese Patent Application No. 2003-300696 filed on Aug. 26, 2003, the content of which is hereby incorporated by reference into this application.

### BACKGROUND OF THE INVENTION

The present invention relates to a chip for processing of gene that processes a gene in a sample that was delivered.

Conventionally, analysis of biological polymers such as gene had problems of requiring complicated steps and a few days' time. Gene analysis may be roughly divided into the pre-treatment step in which a gene of a subject is detected from a sample such as blood and the detection step in which a, gene sequence etc. are analyzed. A technique to automate one of the steps on one cartridge has been disclosed in WO 99/33559, which illustrates an example in which a sample is delivered into a cartridge containing reagents, the gene is detected during the step of the reagents running in the cartridge, and the gene is amplified by a polymerase chain reaction (gene amplification).

However, since the analytical cartridge described in said known example has mounted thereon a multitude of mechanical parts such as a valve, it is not easy to perform the process of mixing with complicated reagents etc. on the cartridge in an efficient manner. Alternatively, the whole cartridge becomes large due to the large built-in disposal tank, thereby limiting reduction in size. Thus, it is not suitable for the format in which a cartridge is disposed in each test.

Also, the flow of the reagent is large at 1-100 mL, the mixing mechanism of a reagent and a sample is required, which increases the size of the analytical instrument. Furthermore, when temperature control of samples and reagents is to be attempted, their large size hinders good thermal response, which results in problematically long analyzing hours. Furthermore, the cost of reagent also becomes a problem.

Thus, the present invention intends to provide a chip for processing of gene that resolves at least one of the above problems.

### SUMMARY OF THE INVENTION

In order to solve the above problems, the present invention encompasses the following format, by which the processing of gene can be simply carried out on the chip with a small amount of reagent in a short period of time.

(1) A chip for processing of gene that is equipped with an injection port into which a sample containing gene is delivered, a gene extraction part into which a solution containing said sample is introduced and which has a gene-binding carrier that binds to said gene, a washing solution-storing part that stores the washing solution to be introduced into said gene extraction part, and a reaction part into which said gene captured in said gene extraction part is introduced,

wherein a fluid channel through which said washing solution is introduced from said washing solution-storing part has been connected to a region more remote from said injection port than from a region into which a solution containing said sample is introduced in said gene extraction part.

When there is a storing part for the eluting solution, this can also be connected from the side remote from said injection port in said gene extraction part.

Alternatively, it is constructed so that the washing solution that passed through the gene extraction part is discharged to the injection port end. Specifically, it is a chip for processing of gene that is equipped with said injection port, said eluting solution-storing part, said gene extraction part, said washing solution-storing part, and said reaction part, wherein it has been formed so that said washing solution that has been introduced from said washing solution-storing part to said gene extraction part flows into said injection port end after flowing out of said gene extraction part.

Alternatively, it is a chip for processing of gene that is equipped with an injection port into which a sample containing gene is delivered, a gene extraction part into which said sample is introduced and which has a gene-binding carrier that captures said gene, a washing solution-storing part that stores the washing solution to be introduced into said gene extraction part, and a reaction part into which the gene extracted at said gene extraction part is introduced, wherein said injection port, said gene extraction part, and said washing solution-storing part have been arranged in series through a fluid channel.

In these formats, when there is a dissolving solution-storing part that stores the eluting solution to be delivered to the sample, said dissolving solution-storing part, said injection port, said gene extraction part, and said washing solution-storing part can be disposed in series through a fluid channel. When this is the case, the mixture of the dissolving solution and the sample is introduced to the gene extraction part.

A specific embodiment may take the following format. It is a chip that is equipped with an injection port into which a sample containing gene is delivered, a dissolving solution-storing part that stores the dissolving solution to be introduced to said sample that was delivered to said injection port, a gene extraction part into which a mixture of said sample and said dissolving solution is introduced and which has a gene-binding carrier which binds to said gene, a washing solution-storing part that stores the washing solution to be introduced into said gene extraction part, an eluting solution-storing part that stores the eluting solution to be introduced into said gene extraction part, and a reaction part into which said gene eluted from said eluting solution is introduced, and that has a fluid channel which branches from between said injection port and said gene extraction part and is connected to said reaction part. Furthermore, it is preferred to have an amplifying solution-storing part that stores the amplifying solution which is introduced into said reaction part.

By attaining these formats, a waste tank for the washing solution that passed through the gene extraction part etc. can be obviated or reduced in size, leading to overall reduction in size in an efficient manner.

(2) A chip for processing of gene that is equipped with an injection port into which a sample containing gene is delivered, a dissolving solution-storing part that stores the dissolving solution to be introduced into the sample that was delivered into said injection port, a gene extraction part into which a mixture of said sample and said dissolving solution is introduced and which has a gene-binding carrier that captures said gene, a washing solution-storing part that stores the washing solution to be introduced into said gene extraction part, and an eluting solution-storing part that stores the eluting solution to be introduced into said gene extraction part, and wherein either storing part of said dissolving solution-storing part, said washing solution-storing part, and said eluting solution-storing part is formed by connecting, through bending parts, a plurality of fluid channels longer in length in the longitudinal direction than in width, and the other end of said storing part has an introducing part of a fluid to be introduced when said solution stored is discharged from the storing part.

Thereby, the remaining solution in each reagent-storing part can be effectively prevented, and the chip can prevent the extra storage of the remaining reagent, and therefore a small chip can be constructed, thus attaining reduction in size of apparatus that uses the chip. Each of the storing part preferably takes the above-mentioned format.

An example of a specific format can be a fluid channel equipped with a plurality of bends that meander in the storing part.

For example, the cross-section of a fluid channel constituting any of the above-mentioned storing part has a maximum surface area 10 times or less that of the connecting fluid channel that connects said storing part and said injection port.

However, it is preferably a lower limit to the extent that loss of the fluid channel does not become large. For example, 0.5 times or more.

In the storing part in the form of a meandered fluid channel, it is conceived that length in the longitudinal direction as the whole storing part is 10 times or more than width in the longitudinal direction of the tubular fluid channel storing part constituting the storing part. For example, it is preferably about 500 times or less from the viewpoint of pressure loss. Also, the structure of the cross section is preferably such that the ratio of width and length is 10 times or less.

(3) A chip for processing of gene equipped with:

the first fluid introduction part that is equipped with an injection port into which a sample containing gene is delivered, a dissolving solution-storing part that stores the dissolving solution to be introduced into the sample that was delivered into said injection port, a gene extraction part into which a mixture of said sample and said dissolving solution is introduced and which has a gene-binding carrier that captures said gene, a washing solution-storing part that stores the washing solution to be introduced into said gene extraction part, an eluting solution-storing part that stores the eluting solution to be introduced into said gene extraction part, and a reaction part into which said gene eluted with the eluting solution is introduced, and that is located at the side more remote from said injection port than from the region in which said dissolving solution has been stored in said dissolving solution-storing part and in which a fluid is delivered to said dissolving solution-storing part when said dissolving solution is introduced into said injection port,

the first fluid discharge part that discharges the fluid in said gene extraction part out of said gene extraction part before said sample and said dissolving solution are introduced into a region more remote from said injection port than the area into which said sample and said dissolving solution are introduced in said gene extraction part,

the second fluid introduction part into which a fluid is delivered when said washing solution is introduced into said injection port at the side more remote from said gene capturing part than the area in which said washing solution has been stored in said washing solution-storing part,

the third fluid introduction part into which a fluid is delivered when said eluting solution is introduced into said injection port at the side more remote from said gene capturing part than the area in which said eluting solution of said eluting solution-storing part has been stored in said eluting solution-storing part, and the fourth fluid introduction part into which a fluid is delivered when said solution containing said eluted gene is introduced from said gene extraction part into said reaction part.

(4) Preferably either of the chips of said (1)-(3) further has the following format.

For example, a format in which the bottom part of the reactor constituting the reaction part is a piezoelectric element. More preferably, for example, a format in which various nucleotides such as those with known base sequences are immobilized on the surface of the piezoelectric element.

The solution that is to be introduced into the reaction part is preferably about 100 μl or less. More preferably, it is 10-30 μl. Also, 10-20 μl is preferred since it enhances processing efficiency.

In order to enhance the analytical effect, it is preferred to limit reduction in size in order to secure the area of the reaction part larger than the area of the sample-introduction part (seen from the direction of light).

The sample to be delivered may be pre-treated (disrupted hard shell of bacteria).

(5) Preferably, the main body equipped with the chip for processing of gene is equipped with a member that supports the chip (the member is preferably equipped with a fluid channel connecting to the analytical chip, and a groove for adhesion), a fixing mechanism that fixes the chip to said supporting member in a detachable manner, a mechanism (pump) that allows movement in the chip of the reagent contained in the reagent tank by transporting or aspirating a fluid to the reagent tank in the analytical chip through the fluid channel of the substrate and that of the analytical chip, a fluid control mechanism (valve) that opens or closes the fluid channel of the substrate, and a detection part (optical detection such as luminescence, fluorescence, colorimetry, etc., and determination of changes in frequency of the piezoelectric element) that detects the gene in the reactor of the chip.

Specifically, for example, it is controlled so that the washing solution flows from the gene extraction part to the injection port side. Specifically, it is an apparatus for processing of gene which is equipped with a chip mounting part having mounted thereon a chip for processing of gene that is equipped with an injection port into which a sample containing gene is delivered, a gene extraction part into which a solution containing said sample is introduced and which has a gene-binding carrier that binds to said gene, a washing solution-storing part that stores the washing solution to be introduced into said gene extraction part, and a reaction part into which said gene captured in said gene extraction part is introduced, a fluid introduction mechanism that introduces a fluid into said chip for processing of gene, and a detection mechanism that detects the eluted gene, wherein said apparatus is controlled so that said washing solution that was introduced from said washing solution-storing part to said gene extraction part flows from said gene extraction part to said injection port.

Alternatively, it is an apparatus for processing of gene which is equipped with:

a chip mounting part having mounted thereon
a chip for processing of gene that has an injection port into which a sample containing gene is delivered,

a gene extraction part, formed in connection with the injection port, into which a solution containing said sample is introduced and which is equipped with a gene extraction part having a gene-binding carrier that captures said gene, and

a washing solution-storing part formed in connection with said gene extraction part, and

that is equipped with a fluid connection part of the gene extraction part downstream of said gene extraction part relative to said injection port wherein the outside and the fluid are connected, and a fluid connection part of the washing solution-storing part downstream of said washing solution-storing part relative to said gene extraction part wherein the outside and the fluid are connected;

a fluid control mechanism that introduces or aspirates a fluid into said chip for processing of gene; and

a detection mechanism that detects the gene contained in said sample,

wherein, said fluid connection part of the gene extraction part is controlled to permit the flow of a fluid between the inside of said chip and the outside of said chip, and said fluid connection part of the washing solution-storing part is controlled to limit the flow of a fluid between the inside of said chip and the outside of said chip, thereby to control to permit the introduction of a solution containing

said sample from said injection port to said gene extraction part, and said fluid connection part of the gene extraction part is controlled to limit the flow of a fluid between the inside of said chip and the outside of said chip, and said fluid connection part of the washing solution-storing part is controlled to permit the flow of a fluid between the inside of said chip and the outside of said chip, thereby to control to permit the flow of said washing solution from said washing solution-storing part through said gene extraction part to said injection port.

Alternatively, for example, the injection port may be left open by aspirating so that the fluid in said chip for processing of gene flows from the inside of said chip to the outside of said chip in the fluid connection part of said gene extraction part and by limiting the flow of a fluid between the inside of said chip and the outside of said chip in the fluid connection part of said washing solution-storing part, thereby to control to permit the flow of said solution containing the sample from said injection port to said gene extraction part.

The injection port has a wall, in between the injection port and the air, that prevents its communication with the air by allowing a fluid to flow in between the inside of said chip and the outside of said chip in the fluid connection part of said gene extraction part, by limiting the flow of a fluid in between the inside of said chip and the outside of said chip in the fluid connection part of said washing solution-storing part, by delivering a fluid to said injection port, and by controlling to permit the flow of said solution containing the sample from said injection port to said gene extraction part.

It is also equipped with means to control the temperature of the reactor in the analytical chip. It is preferred that a predetermined temperature cycle is applied to amplify the gene.

Alternatively, a format may be used in which gene is amplified at a temperature range of 60-65° C. and fluorescence is detected or opaqueness due to the byproduct magnesium pyrophosphate is determined by photometry. Or, when a gene is bound to the surface of a piezoelectric element to which a nucleotide whose base sequence is known has been bound, oscillating frequency of the piezoelectric element changes. By determining this change in frequency, the sequence of the gene complementary to the immobilized nucleotide can be read.

(6) It is a method of using a chip for processing of gene comprising the steps of cooling and freezing a chip for processing of gene having a sample injection port into which a test sample is injected, a reagent tank, in connection with said sample injection port, in which reagents have been stored, a fluid channel for extracting gene from said test sample, a reactor in which the extracted gene is detected, and a fluid channel connecting said reagent tank and the external fluid channel, and of carrying said frozen chip for processing of gene. Alternatively, refrigeration in stead of freezing may also be conceived.

(7) It is a method of detecting gene comprising the steps of providing a chip for processing of gene having a sample injection port for injecting a test sample containing a gene, a reagent tank, connected to said sample injection port, in which reagents have been stored, a fluid channel for extracting gene from the test sample, a reactor for detecting the extracted gene, and a fluid channel connecting said reagent tank and the external fluid channel after the chip was once cooled, refrigerated or frozen; bringing the provided analytical chip back to room temperature; introducing a sample containing the gene into said sample injection port and extracting gene from said reagent with said reagent; and detecting said gene.

By using these formats, without mounting a multitude of mechanical parts such as a valve, the chip for processing of gene can efficiently perform complicated processing of mixing of reagents etc. on the chip. Alternatively, by storing the used waste in the injection port etc., overall reduction in size can be attained, which is suitable for the format in which cartridges are disposed in each test.

Also, by minimizing the flow of reagent, the reagent and the sample can be effectively mixed leading to possible reduction in size, or the reagent and the sample have high thermal response which effects temperature control, thereby enabling curtailment in the time required for analysis.

In accordance with the present invention, there can be provided a chip for processing of gene or an apparatus for processing of gene that permits simple processing of gene on the chip in a short period of time even with a trace amount of reagent.

The purposes, characteristics and advantages of the present invention will be apparent from the following description of the present invention with reference to the attached drawings.

### BRIEF DESCRIPTION OF THE SEVERAL VIEWS OF DRAWING

FIG. 1 is an enlarged view showing a constitution of an analytical chip.

FIG. 2 is a flow chart showing a procedure of making a reaction chip.

FIG. 3 is a cross-sectional view of an analytical chip of Working Example 1.

FIG. 4 is a cross-sectional view showing a constitution of an analytical chip.

FIG. 5 is a flow chart showing an analytical procedure of Working Example 1.

FIG. 6 is a drawing showing a profile of fluid handling of Working Example 1.

FIG. 7 is an example of the result of experiment of Working Example 1.

FIG. 8 is a cross-sectional view of an analytical chip of Working Example 2.

FIG. 9 is a drawing showing a profile of fluid handling of Working Example 2.

### DETAILED DESCRIPTION OF THE INVENTION

Examples of the present invention will now be explained below. The present invention is not limited to the disclosure of the specification nor does it limit modifications based on known technology.

As an embodiment of the present invention, an example is explained whether the presence or absence of the subject gene can be detected by extracting a gene from a sample and then amplifying the gene by a polymerase chain reaction. As used herein, a sample may be blood, bacteria, viruses and the like.

(Principle of Analysis)

The extraction of gene may be effected by a conventionally known solid-phase extraction method. The solid-phase extraction method is a method in which a gene is first allowed to specifically bind to the surface of a solid, and then the gene alone is eluted separately from other substances into an aqueous solution for extraction.

First Stage—Lysis of Cell Membrane

A sample is mixed with a solution containing a caotropic ion (a monovalent anion having a large diameter of the molecule) in order to destroy viruses or cell membrane covering the subject gene by the action of a caotropic ion. The caotropic ion also serves to denature various proteins contained in the sample and to inhibit the action of nucleases (enzymes that decompose nucleic acids).

Second Stage—Binding

When silica is added to the mixture after lysis, gene and silica specifically bind to each other by the action of the caotropic ion. Generally, a method is employed in which the mixture is passed through a glass filter.

Third Stage—Washing

Since proteins contained in the sample and caotropic ions that contaminated the extract may inhibit the detection of gene by gene amplification, a procedure of washing gene-silica is required. A high concentration of ethanol is used in washing herein. Since gene is hardly soluble in a high concentration of ethanol, the gene adsorbed to the silica cannot be eluted in this process.

Fourth Stage—Elution

After washing, water and a solution of low concentration salt are added to the gene-silica in order to elute the gene from the silica.

Fifth Stage—Gene Detection

To the eluted gene, a primer (a single stranded DNA having the same base sequence as about 20 bases on both ends of the DNA of interest), a DNA synthetase (polymerase) and four types of substrates (dNTP) etc. are added, and the gene is subjected to a temperature cycle “heat denaturation-annealing-synthesis of complementary strands” for amplification (polymerase chain reaction). Here, in addition to said reagents, a fluorescent dye may previously be injected and the temperature cycle may be applied under the irradiation of an excitation light in order to detect gene amplification on a real time basis.

(Constitution of Analytical Chip)

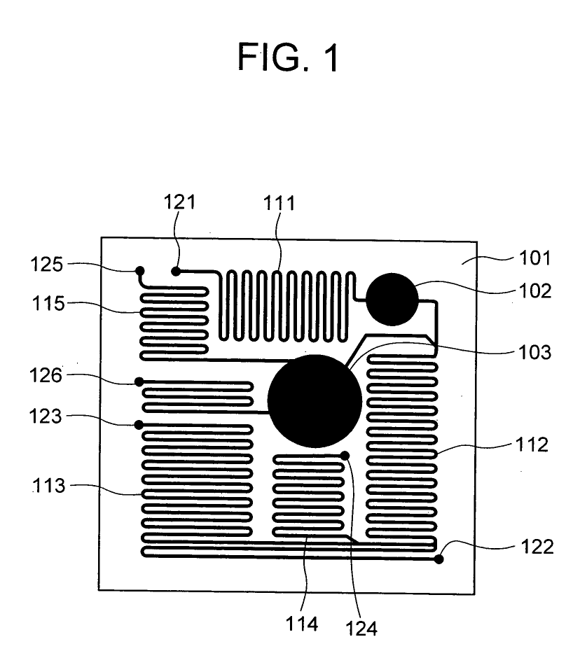

The constitution of an analytical chip for processing of gene is explained with reference to FIG. 1. FIG. 1 is a detailed drawing of an analytical chip 101. In this Working Example, a format equipped with a dissolving solution-storing tank and reagents for amplifying gene is explained.

The analytical chip 101 comprises a dissolving solution-storing tank 111 that stores the cell membrane-dissolving solution, a sample injection port 102 (this may optionally be used as a waste tank), a gene extraction area 112 in which a gene-binding carrier has been filled in the fluid channel, a washing solution-storing tank 113 that stores the washing solution, an eluting solution-storing tank 114 that stores the gene eluting solution, a reactor 103 that performs the detection of gene, chip ports (121-126) that are located at a further end of the fluid channel than a position in which the stored solution is located in the fluid channel constituting each tank and that serve as a contact with the external fluid channel. The fluids inside and outside of the CHIP are communicated therein. A fluid is for example a gas such as the air. Optionally, it may be a solution such as water. This Working Example further illustrates an example which is further equipped with a gene amplification reagent-storing tank 115 that stores reagents for gene amplification. In these chip ports, the inside and the outside of the chip can be communicated by a fluid. The majority of these chip components are micro fluid channels pattern-copied by the microfabrication technology.

The method of making an analytical chip 101 is described here. As a material for an analytical chip 101, a resin having excellent disposability is more preferred than glass that is expensive in processing and fragile. The type of resin used is, but not limited to, polydimethylsiloxane (PDMS: manufactured by Dow Corning Asia, Sylpot184) having the following excellent characteristics. The chip is preferably equipped with the following characteristics:
Excellent biocompatibility (ordinary silicone rubber is physiologically inert)
Copying of pattern can be effected with a submicron precision (before curing, it has low viscosity and high fluidity, and thus can favorably permeate into details of complicated shapes)
Low cost (8 yen/gram. Less than a one-hundredth of that of conventional general-purpose material, Pyrex glass, for microdevices which is 1000 yen/gram)
Easily disposable by incineration

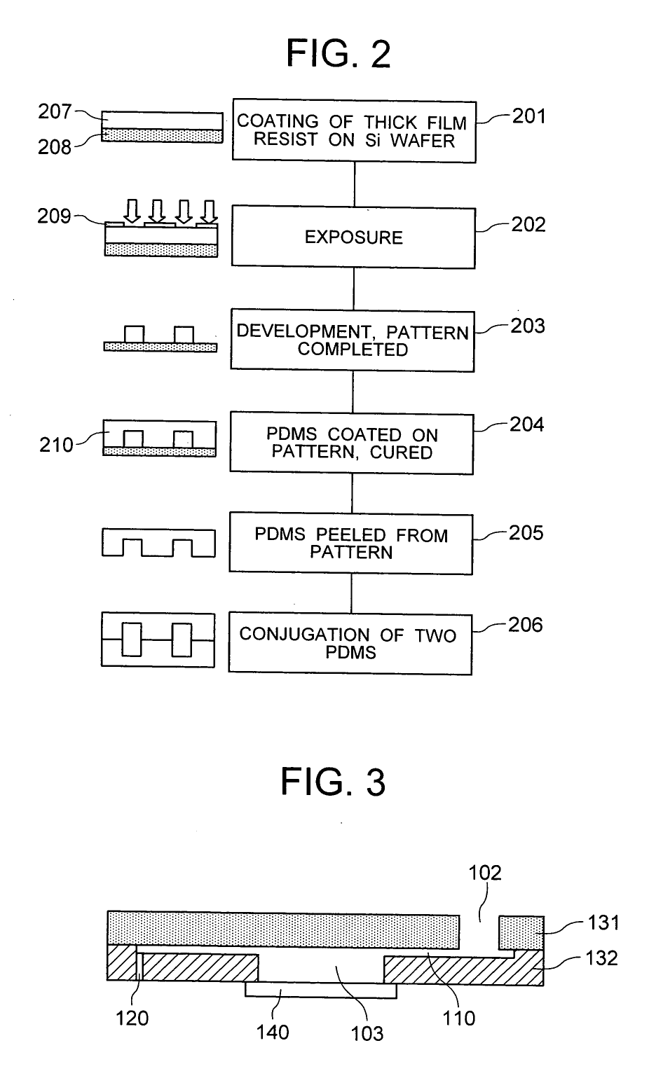

FIG. 2 shows a flow of making an analytical chip 101 using a resin substrate (the following illustrates an example in which PDMS was used). An analytical chip can be molded by constructing a pattern molding components of the analytical chip by the photolithography technology, followed by copy-molding of the pattern onto a resin. The process can be roughly divided into [1] molding of a pattern to be copied to PDMS, [2] pattern copying to PDMS, and [3] conjugation between PDMS.

[1] Molding pf a Pattern to be Copied to PDMS

A micro pattern can be molded by the steps of coating a photosensitive thick film resist 207 (manufactured by Micro. Chem., NANO SU-8) as a pattern material on a silicon wafer 208 (step 201), exposing by placing a photomask 209 on a photosensitive thick film resist 207 (step 202), and developing images (step 203). Unlike photofabrication by a conventional wet etching, it has an advantage that a curved shape can be molded while retaining a rectangular cross section.

[2] Pattern Copying to PDMS

PDMS 210 and a curing agent are mixed at a weight ratio of 10:1, coated on the pattern, and heated at 100° C. for 1 hour so that PDMS 210 is cured (step 204). From a convex micropattern, a concave PDMS 210 can be obtained (step 205).

[3] Conjugation Between PDMS

The surface of the pattern-copied PDMS 210 was treated with oxygen-plasma, and two sheets of PDMS 210 are superimposed to conjugate two sheets of PDMS 210. The strength of conjugation is sufficient so that when force is applied to peel the conjugation site it will break PDMS 210. One of the two may be PDMS 210 and conjugated to silicone or glass. The method of molding PDMS is not limited to said method, and for example an extrusion molding can be used for processing.

A more detailed structure of the analytical chip 101 is explained with reference to FIG. 1 and FIG. 3. FIG. 3 is a cross-sectional view of an analytical chip 101. The analytical chip 101 is two sheets of PDMS that were plasma-treated to reform the surface and were conjugated. First, in the PDMS first layer 131, there has been formed a through-hole that serves as a sample injection port and a waste tank 102. The volume of the sample injection port and a waste tank 102 is 50-100 μl and mostly open to the air. In the PDMS second layer 132, micro fluid channels 110 have been pattern-formed that serve as various reagent tanks (111-115). The micro fluid channels 110 were formed with the shape of the cross section being 0.1 mm long and 0.5 mm wide. The shape of the cross section is not specifically limited, and the width/length of 10 or less is preferred. If the width/length is 10 or greater, the PDMS first layer 131 may bend and thereby to break the rectangular structure of the micro fluid channels 110.

Furthermore, in the PDMS second layer 132, there have been formed a chip port 120 that communicates the micro fluid channels 110 and the fluid channel of the external apparatus and a through-hole that serve as a reactor 103. The volume of the solution containing the gene to be introduced into the reactor 103 is 10-30 μl Specifically by retaining the volume of the reactor 103 at 10-30 μl thermal responses become enhanced and temperature control of the reactor 103 becomes rapid. This enables the progress of reaction at an optimum condition while changing the temperature of the reactor 103 in seconds. Also, by minimizing the volume of the reactor 103, mixing of the eluting solution and the reagent for gene amplification may be effected in a shorter period of time (for example about one second), thereby simplifying the mixing procedure. The reactor 103 has a larger surface area seen from the direction of thickness than the sample injection port 102. In the conventional technology (WO 99/33559), an ultrasonic element etc. was used for mixing, but according to the present invention, it is possible that the mixing procedure is not used for simplification. Alternatively, if used, it is simple.

Since gene is detected in the reactor 103, a plate that provides the base is required. As described below, since the base plate 140 of the analytical chip 101 also plays a role as a medium for transferring heat from the temperature control mechanism to the reactor 103, it is preferably a material having a good thermal conductivity. Furthermore, if the surface of the base plate 140 is mirror surface, fluorescence in the reactor 103 is reflected at the base plate 140, the sensitivity of detecting gene becomes high. What is preferred as the base plate 140 is chromium, and most preferably silicon. Because silicon has a good thermal conductivity and can be easily conjugated with PDMS with the oxygen-plasma treatment alone.

The analytical chip 101 of the present invention has built in four reagent tanks (a dissolving solution-storing tank 111, a washing solution-storing tank 113, an eluting solution-storing tank 114, a gene amplification reagent-storing tank 115). All of the reagent tanks preferably has the shape of fluid channel. In order to transport the reagent in a reagent tank, a fluid (air or water) is delivered from behind the reagent tank. Because, if the reagent tank is not of the shape of fluid channel at this time, the reagent at the location (a site having a favorable solution wettability) in which a solution can easily pass through is only extruded, and reagents at other sites remain in the reagent tank. In order to reduce the amount of reagent to be consumed, it is effective to render the reagent tank a fluid channel shape.

Preferably the volume of the dissolving solution-storing tank 111 is 10-20 μl, that of the washing solution-storing tank 113 is 10-30 μl, that of the eluting solution-storing tank 114 is 5-10 μl, and that of the gene amplification reagent-storing tank 115 is 5-10 μl. In particular, the sum of the eluting solution (containing gene) and the gene-amplification reagent being 10 μl or less, as described above, is optimum since mixing of the eluting solution and the gene-amplification reagent could become rapid, and the reaction could become homogeneous. Thus, minimizing the volume of the reagent tank and the reactor is advantageous because the volume of reagents can not only be reduced and become low cost, but temperature control becomes rapid, mixing becomes rapid, and reaction becomes homogeneous.

As a gene-binding carrier filled in the gene extraction area 112, quartz wool, glass wool, glass fiber and glass beads can be used. When glass beads are used, the size of beads is preferably 20-50 μm in order to maximize the contact area, and 20-30 μm is most preferred.

In order to hold back the gene-retaining carrier, it is preferred that the fluid channels constituting the area become narrower at one or more sites.

For example, in order to retain the gene-binding carrier in the gene-extracting area 112, the width of the micro fluid channels of the gene-extracting area 112 is narrowed to 10 μm at two sites. Thus, the narrowed fluid channel provides a weir. If the fluid channels become less than 10 μm, fluid resistance becomes greater and thus fluid control becomes difficult. Thus, the width of fluid channel as weir is preferably 10-20 μm.

(Constitution of Analytical Apparatus)

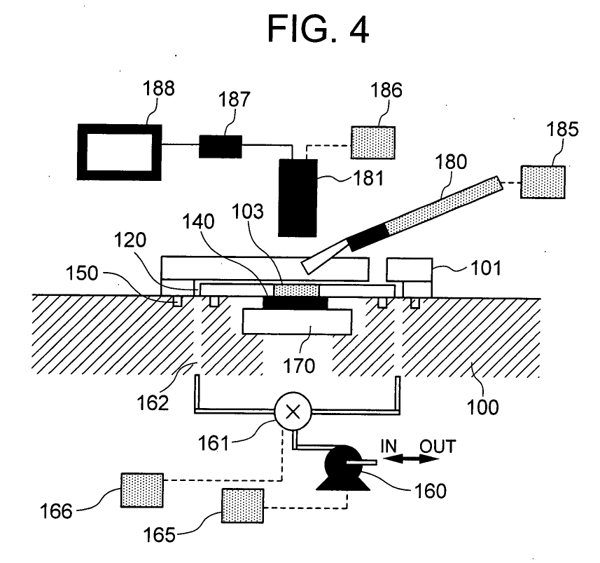

FIG. 4 shows a cross-sectional view of an analytical apparatus in which an analytical chip 101 is set. The present analytical apparatus is roughly divided into three systems: the fluid system, the temperature control system, and the optical detection system. The substrate 100 on which the analytical chip 101 is set has built in an adhesion groove 150 for adsorbing the analytical chip 101, an in-apparatus fluid channel 162 in communication with a port of the analytical chip 101, and a temperature control mechanism 170 for optimizing the temperature of the reactor 103. The analytical apparatus has control mechanisms for performing each control. On the analytical apparatus are mounted a pump control mechanism 165 that controls a pump 160, a valve control mechanism 166 that controls a valve 161, a light source control mechanism 185 that controls a light source 180, a photodetector control mechanism 186 that controls a photodetector 181, a light signal transformer 187 that transforms a signal of the photodetector, and a data display screen 187 that displays the transformed light signals.

By placing an analytical chip 101 on the substrate 100 and vacuum aspirating the adhesion groove 150, the analytical chip 101 adsorbs to the substrate 100. By performing vacuum chuck in this way, the chip port 120 is securely connected to the in-apparatus fluid channel 162 to prevent solution leakage, while the analytical chip 101 becomes easily detachable from the substrate 100. In order to render the analytical chip 101 disposable, the fixing method of the analytical chip 101 by vacuum chuck is very practical.

As the temperature control mechanism 170, various heating elements can be applied, and preferably, for example, it is a Peltier device. When a Peltier device is used, the procedure of temperature rise and cooling of the reactor 103 can be performed simply by changing the direction of the applied electric current.

The in-apparatus fluid channel 162 is each connected to the pump 160 through the valve 161. The pump 160 is preferably one that can switch perflation and aspiration, and preferably there are a plurality of them. If transporting of a reagent in a reagent tank is desired, the valve 161 is switched so that perflation is only effected to the port in communication with the reagent tank.

The valve 161 that controls fluids in this way is preferably placed at the analytical apparatus side rather than inside of the analytical chip 101. By so doing, the analytical chip 101 becomes free of mechanical parts, thereby attaining size-reduction and disposability.

The optical detection system is composed of a light source 180 that irradiates an excitation light to the gene in the reactor 103, and a photodetector 181 that measures fluorescence from inside of the reactor 103. For example, measurement may be effected with time. As the light source 180, those in different wavelength regions may be used, and when a common ethidium bromide is used as a fluorescent dye, a UV lamp or a UV laser is preferably used. The photodetector 181 is disposed so that the light-receptive surface becomes directly above the reactor 103. As photodetectors 181, there can be mentioned preferably a CCD camera, a photomultiplier tube, a photodiode etc., with the photodiode being preferred in order to reduce the size of the apparatus.

In accordance with the present invention, a large analytical cartridge as in the conventional technology is not required, and there is provided a small and portable analytical apparatus that can be made by placing small analytical chips having no built-in mechanical parts on a substance which are simply assembled.

(Analytical Procedure)

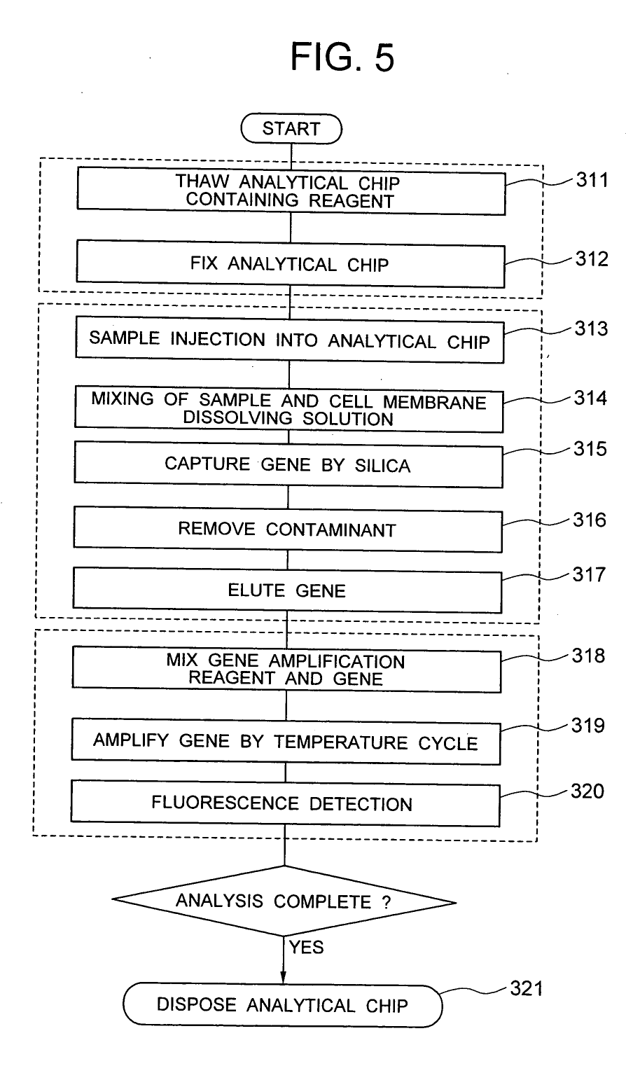

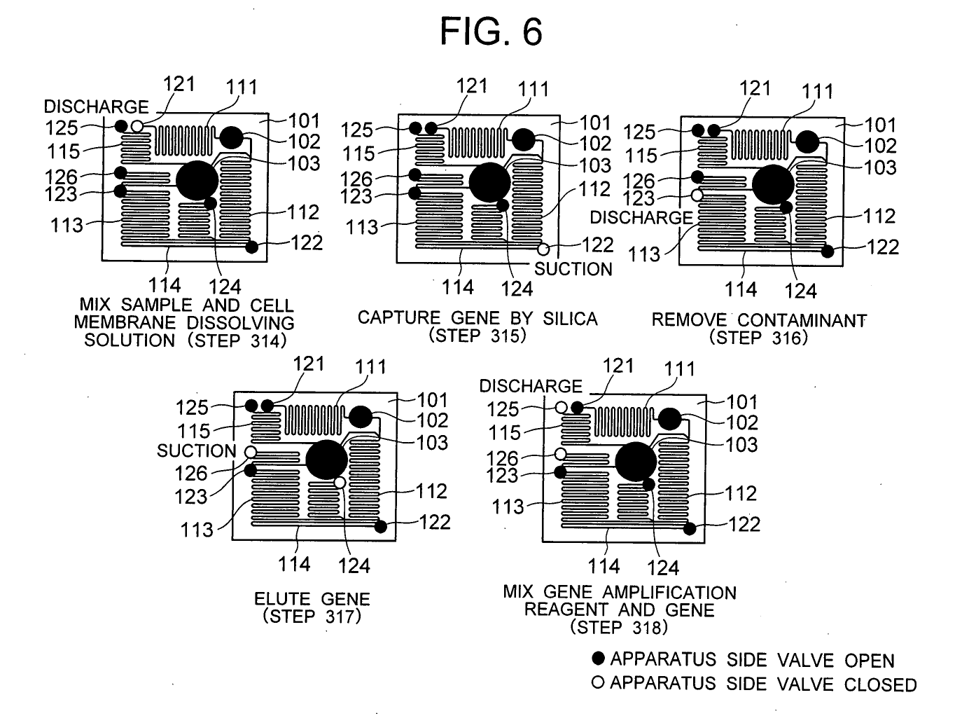

A procedure of analysis using the analytical chip 101 is explained with reference to FIG. 4, FIG. 5, and FIG. 6. FIG. 5 is a flow chart showing the procedure of an analytical method. FIG. 6 is a drawing showing a profile of fluid handling of Working Example 1.

The analytical procedure can have the following procedure.

When it is desired to provide a dissolving solution that destroys the cell membrane of the sample to the sample, a dissolving solution that destroys the cell wall of the sample is first mixed with the sample. When this step is not required, the following procedure is followed directly after the introduction of the sample.

Then, the mixture of said dissolving solution and the sample is transported to a fluid channel filled with a gene-retaining carrier.

Then, a washing solution that washes proteins etc. contained in the sample is transported to a fluid channel filled with said gene-retaining carrier, and the waste solution is transported to a tank in which the sample had originally been retained.

Then, an eluting solution that elutes the gene adsorbed to the gene-retaining carrier is transported to a fluid channel filled with said gene-retaining carrier, and further transported to a reactor in which the gene is detected.

Then, the presence of the subject gene is detected. An example is specifically explained below.

First, the analytical chip 101, that had been frozen, having built-in a dissolving solution-storing tank 111, a washing solution-storing tank 113, an eluting solution-storing tank 114, and a gene amplification reagent-storing tank 115, each containing each of four types of reagents, i.e. a cell membrane dissolving solution, a washing solution, a gene eluting solution, a gene-amplification reagent, respectively, is thawed at room temperature. By providing with the user reagent for one test previously contained in an analytical chip 101, the analytical chip 101 can be rendered a single-use without the waste of the reagent, and economy is increased, Also the user can obviate the labor of delivering reagents into each reagent storing tank, which can not only shorten time, but can prevent contamination. Furthermore, by providing with the user this analytical chip 101 at a frozen state and by the user's storing the analytical chip 101 frozen at 0° C., the activity of the reagent can be maintained for one month. Also by storing frozen at −20° C., the activity of the reagent can be maintained for half a year or longer. Thus, by providing with the user the disposable analytical chip 101 having built-in reagent for one test in advance at a frozen or refrigerated state, a simple analytical environment can be created (step 311).

Then, the analytical chip 101 is placed on the substrate 100, and after confirming the communication of the chip port 120 and the in-apparatus fluid channel 162, the adhesion groove 150 is reduced in pressure. By producing a vacuum in this way, the analytical chip 101 is fixed on the substrate 100 by a vacuum chuck (step 312). Then, a 10 μl of the sample is delivered to the sample injection port and a waste tank 102. The sample is a sample containing gene, and is blood, bacteria, virus etc. (step 313)

Subsequently, by switching the valve 161 in the analytical apparatus, a fluid is run from the pump 160 only to the dissolving solution port 121 (dissolving solution port 121: open, other ports 122-126: closed). The fluid used here may be any of water, alcohol, air, etc. unless it deteriorates the activity of the reagent when contacted with the reagent. The 20 μl of the cell membrane dissolving solution in the dissolving solution-storing tank 111 is injected to the sample injection port and a waste tank 102 by a fluid, and mixed with the sample in the sample injection port and a waste tank 102. This disrupts the cell membrane releasing the gene of the sample out of the cell. A preferred cell membrane dissolving solution is a caotropic ion solution containing guanidine thiocyanate, guanidine hydrochlorate, sodium iodide, potassium bromide or the like (step 314).

Subsequently, by switching the valve 161 in the analytical apparatus, the aspiration of the pump 160 is started so as to evacuate the inside of the chip from the chip port A 122 (chip port A 122: open, other ports 121, 123-126: closed, the dissolving solution port 121 may be open). By this procedure, the gene suspension in the sample injection port and a waste tank 102 moves to the gene-extracting area 112. When all of the gene suspension in the sample injection port and a waste tank 102 has moved to the gene-extracting area 112, the pump 160 is stopped. By so doing, the gene is captured by the gene-binding carrier in the gene-extracting area 112 (step 315).

Subsequently, by switching the valve 161 in the analytical apparatus, the chip port A 122 is closed and the fluid is run from the pump 160 only to the washing solution port 123 (washing solution port 123: open, other ports 121-122, 124-126: close, the dissolving solution port 121 may be open). 20 μl of the washing solution in the washing solution-storing tank 113 is transported by a fluid to the gene-extracting area 112. As the washing solution, Tris-HCl can be used, and ethanol at a high concentration of 50% or higher is more preferred. With this washing solution, proteins and caotropic ions remaining in the gene-extracting area 112 can be eliminated. The washing solution from the gene-extracting area 112 is allowed to run to the sample injection port and a waste tank 102 side. For example, when the washing solution that washed the gene-extracting area 112 has moved to the sample injection port and a waste tank 102, the pump 160 is stopped. In the conventional example (WO 99/33559), the waste reservoir is too large and as a result the analytical cartridge was a large-size, but by allowing the waste tank to serve as a sample injection port as in the present invention, the size of the analytical chip 101 can be reduced, which is more suited for the purpose of disposability. In addition to, or in stead of, the sample injection port 102, at this time, it is also possible to introduce the used washing solution into the sample injection port 102. By so doing, the waste can be stored without making the sample injection port 102 a large-size. Alternatively, it is possible to prevent effectively the leakage of the waste from the sample injection port to the outside. At this time, by arranging the dissolving solution-storing tank 111, the sample injection port and waste tank 102, the gene-extracting area 112, and the eluting solution-storing tank 114 in series, the control procedure of fluids can be most simplified and the time for analysis can be minimized (step 316).

Subsequently, by switching the valve 161 in the analytical apparatus, the washing solution port 123 is closed, and the elution port 124 and the chip port B 126 are opened (the elution port 124, the chip port B 126: open, other ports 121-123, 125: closed, the dissolving solution port 121 may be open). 5 μl of the eluting solution in the eluting solution-storing tank 114 is transported to the gene-extracting area 112 by a fluid. As the eluting solution here, sterilized distilled water, a buffer solution such as TRIS-EDTA and TRIS-acetate can be used. With this eluting solution, the gene that had been captured to the gene-binding carrier in the gene-extracting area 112 is eluted. When the eluting solution containing the gene reached the end of the gene-extracting area 112, the aspiration of another pump 160 is started so as to evacuate the reactor 103 from the chip port B 126. By so doing, the eluting solution containing the gene is guided to the reactor 103 without being transported to the sample injection port and a waste tank 102. When all of the eluting solution has moved to the reactor 103, the pump 160 is stopped. By so doing, the sample pretreatment or the extraction of the gene is complete (step 317).

Then, the extracted sample is subjected to detection by a gene detection apparatus.

The following is an example of gene detection procedure. By switching the valve 161 in the analytical apparatus, the eluting solution port 124 and the chip port B 126 are closed, and the fluid is run only to the gene amplification reagent port 125 from the pump 160 (the gene amplification reagent port 125: open, other ports 121-124, 126: closed, the dissolving solution port 121 and the chip port B 126 may be open). Five μl of the gene-amplification reagent in the gene amplification reagent-storing tank 115 is injected to the reactor 103, and mixed with the gene in he reactor 103. The gene-amplification reagent is composed of 2.5 mM of four types of dNTP (dATP, dCTP, dGTP, dTTP), a buffer (100 mM Tris-HCl, 500 mM KCl, 15 mM MgCl2), two types of primers, DNA synthetases (either of Taq DNA polymerase, Tth DNA polymerase, Vent DNA polymerase, and thermosequenase), and a fluorescent dye (either of ethidium bromide, and SYBR GREEN (manufactured by Molecular Probe)) (step 318).

Then, the temperature control mechanism 170 mounted at the bottom of the analytical chip 101 is driven, and a temperature cycle is applied so that the temperature of the reactor 103 shuttles to the following two predetermined values through the base plate 140 (step 319).

As an example of temperature cycle, roughly the following may be performed: [90-95° C. for 10-30 seconds ⇄65-70° C. for 10-30 seconds]×30-45 times

As a preferred example, the following temperature cycle is performed: [94° C. for 30 seconds ⇄68° C. for 30 seconds]×45 times

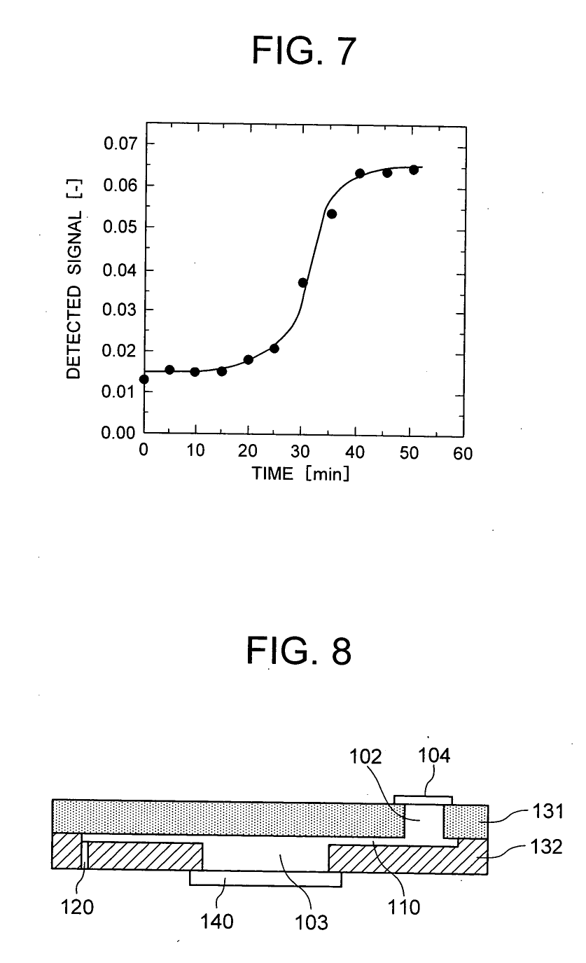

While performing the temperature cycle, an excitation light is irradiated to the reactor 103 from the light source 180 on top of the analytical chip 101. The gene, if it has a fluorescent dye intercalated into the inside of the double strand, transfers the energy of the absorbed light of the light source 180 to a fluorescent dye (energy transfer). As a result, the fluorescent dye is excited and emits fluorescence. Thus, when the gene of interest is present in the sample, the amount of fluorescence emitted increases as the gene is amplified. Therefore, by monitoring the amount of fluorescence in the reactor 103 by the photodetector 181 during the temperature cycle, the presence or absence of the gene of interest can be detected on a real-time basis as shown in FIG. 7 (step 320).

Then, after lowering the adhesive force of the adhesion groove 150 on the substrate 100, the chip is removed from the substrate. For example, when the analysis is complete, the analytical chip 101 is removed from the analytical apparatus and discarded (step 321). This obviates the need of the post-treatment of the samples and the reagents and the need of the washing procedure of the reaction detection part, and thus can provide simple and rapid analysis.

By using the analytical chips of the present invention, the step of extracting the gene from the sample can be automated in a small chip. Since space-saving was accomplished by the absence of mechanical parts such as valves in the analytical chip and the waste tank that also serves as a reagent injection port, analytical chips suitable for disposability can be provided. Furthermore, as a result of miniaturization of the volume of the reactor and the fluid channels by microfabrication, such advantages can be obtained as reduction in the amount of reagents and in cost as well as rapid temperature control, rapid mixing, and homogeneous reaction. Furthermore, since the reagent for one test is previously contained in a disposable analytical chip and the analytical chip is provided to the user at a refrigerated or frozen state, an extremely simple and fast detection of gene can be attained.

Furthermore, the reactor 103 described in the present example can take a shape equipped with a wall, in between the tank and the air, that prevents communication with the air. On the other hand, from the manufacturing standpoint, the region of the reactor 103 can be open to the air. The number of the reactors is one in the example shown. However, it may be more than one depending on the subject to be analyzed and the like.

By constructing in this way, furthermore, the flow of the fluids and the reagents in the analytical chip has been controlled by the fluid devices (pump, valve), it is possible to make a simple chip construction that can suppress the placement of pumps and a multiplicity of valves in the chip.

Thus, steps to gene extraction from the sample and analysis can be simplified and become rapid, and there can be provided an analytical chip that can contain reagents in advance and can be disposed after analysis together with the reagent, and an analytical apparatus that is equipped therewith.

Furthermore, in accordance with the present invention, by making the cross section of the solution storing tank in the form of a meandering fluid channel and the tubules of the gene extraction area larger than the connecting fluid channel part connecting these storing tanks and other area (for example, injection port and reactor), pressure loss accompanied by the discharge of fluids such as reagents from the storing tank can be minimized.

On the other hand, the size of the cross section of the tubules of the solution storing tank in which the solution has been stored is preferably small to the extent that it can prevent the occurrence of solution remaining at the time of discharge of the solution. For example, it is conceived that the size is about 10-times or less that of the cross section of the tubule of the connecting fluid channel part. Alternatively, from a viewpoint of minimizing loss of expansion or shrinkage of the solution flow between the storing tank and the connecting fluid channel, 5-times or less may be conceived.

The above was defined as the cross section, but it is preferred from the viewpoint of ease in manufacturing that width changes more greatly than the difference in the height without changing greatly the difference in the height of the tubule in the region of the storing tank etc. and of the connecting fluid channel. For example, said values defined as the cross section can be defined as width.

Furthermore, it is preferred that there is an area which is narrower than the cross section of the tubule of said storing tank or the gene extraction area in between the storing tank or the gene extraction area and the corresponding port, because it can prevent the leakage of the stored solution.

[Working Example 2]

The present Working Example can essentially take the shape described in Working Example 1, but in accordance with the present Working Example, the sample injection port and a waste tank 102 of the analytical chip 101 is not open to the air, and at least after the sample has been delivered to the sample injection port and a waste tank 102, a cover such as a wall that prevents communication with the air is formed in the sample injection port 102. For example, a sample injection port and a waste tank cover 104 of a glass thin plate (for example, a cover slip for microscope) etc. having a good adhesion with resins is covered over the sample injection port and a waste tank 102 to seal the sample injection port and a waste tank 102. The step of covering the sample injection port and a waste tank 102 may be manual, but it is more preferred to be equipped with a mechanism of mounting a sample injection port and a waste tank cover 104 on the analytical apparatus side. It is efficient in terms of handling that these covers are a shape that is covered in advance in order to prevent communication with the air.

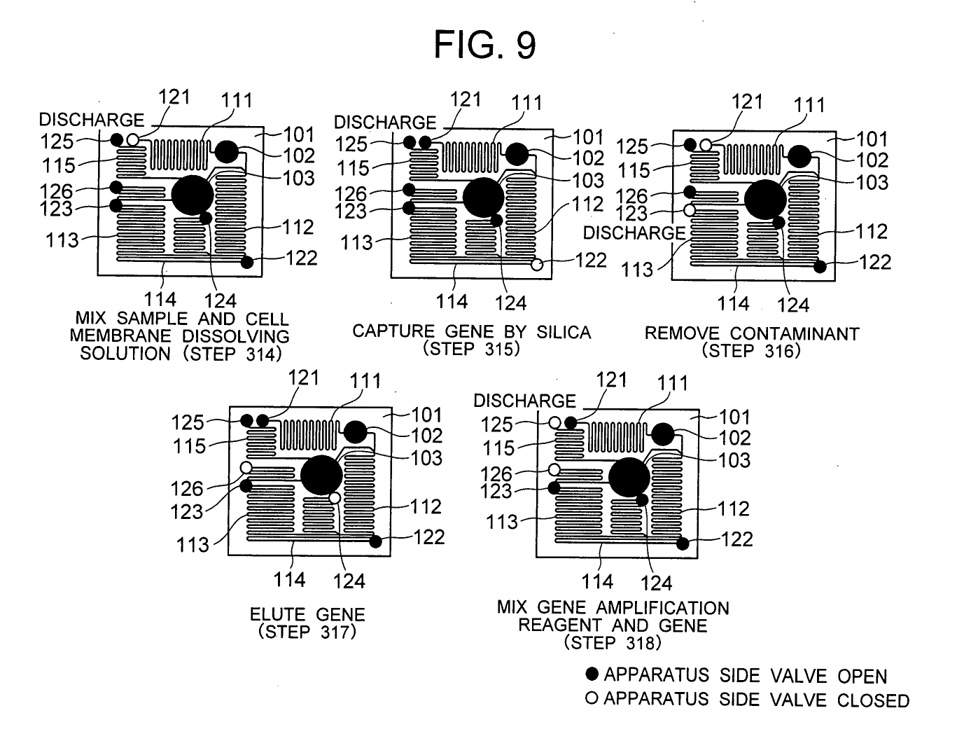

Accordingly, an example of the profile of the fluid handling of Working Example 2 is shown in FIG. 9. Steps 311 to 314 in Working Example 1 is the same in Working Example 2. Step 315 and after are explained. By switching the valve 161 in the analytical apparatus, the chip port A 122 is opened and a fluid is run from the pump 160 subsequently to the dissolving solution port 121 (dissolving solution port 121, chip port A 122: open, other ports 123-126: closed). By so doing, the gene suspension in the sample injection port and a waste tank 102 moves to the gene-extracting area 112. When the transfer of the gene suspension in the sample injection port and a waste tank 102 to the gene-extracting area 112 is complete, the pump 160 is stopped (step 315).

Subsequently, by switching the valve 161, the chip port A 122 is closed, and the fluid is run to the washing solution port 123 while keeping the dissolving solution port 121 open (dissolving solution port 121, washing solution port 123: open, other ports 122, 124-126: closed). The washing solution in the washing solution-storing tank 113 that washed the gene-extracting area 112 is transported to the gene-extracting area 112. When all of the washing solution that washed the gene-extracting area 112 has moved to the sample injection port and a waste tank 102, the pump 160 is stopped (step 316).

Subsequently, by switching the valve 161, the dissolving solution port 121 and the washing solution port 123 are closed, and the elution port 124 and the chip port B 126 are opened (the elution port 124, the chip port B 126: open, other ports 121-123, 125: closed). The pump 160 is driven to run the fluid to the elution port 124. .The eluting solution in the eluting solution-storing tank 114 is transported to the gene-extracting area 112. This eluting solution elutes the gene that was captured by the gene-binding carrier in the gene-extracting area 112, and is guided to the reactor 103 in which the chip port B 126 is open. When all of the eluting solution has moved to the reactor 103, the pump 160 is stopped (step 317).

Thus, by sealing the sample injection port and a waste tank 102, processing can be effectively performed by the inflow procedure of a fluid (for example the air) by a pump. Furthermore, it is possible to prevent contamination from the air that can take place because the sample injection port and a waste tank 102 is open to the air and leakage of reagents.

[Working Example 3]

The present Working Example can essentially take a forma described in Working Example 1, but according to the present invention gene is amplified while keeping the temperature constant.

Steps to the extraction of gene from the sample is the same as in Working Example 1. In this case, the components of the gene-amplification reagent are different. Thus, The gene-amplification reagent is a mixture of 10 mM of four types of dNTP (dATP, dCTP, dGTP, dTTP), a buffer (2 mM of MgSO₄), four types of primers, 100 mM of MgSO₄, 4 M of BETAINE, DNA synthetase (4 units/μl of Bst polymerase), and a fluorescent dye (either of ethidium bromide, and SYBR GREEN (manufactured by Molecular Probe)). The temperature control of the reactor 103 by the temperature control mechanism 170 is in the range of 60-65° C. When the gene of interest is present, the amount of fluorescence increases in about one hour after the start of temperature control. In stead of detecting fluorescence, opaqueness due to the byproduct magnesium pyrophosphate may be determined by photometry.

The designing of four types of primers is somewhat difficult, but due to the absence of temperature cycle, temperature can be controlled by a heater simpler than Peltier.

It has an advantage that the components of the analytical apparatus can be simplified.

[Working Example 4]

The present Working Example can essentially take a forma described in Working Example 1, but as the base plate 140 of the analytical chip 101, a piezoelectric element such as a quartz oscillator or a surface acoustic wave element is applied. Since the piezoelectric element changes the weight applied on the electrode to changes in oscillating frequency in a quantitative manner, it has been widely used as a tool for determining minute changes in weight under a reaction atmosphere on a continuous basis. Thus, various nucleotides of which base sequences are known are fixed on the piezoelectric element as a base plate 140. The method of fixing is preferably as follows: First, a glass thin film is formed on the electrode of the piezoelectric element by sputtering, vapor deposition, and the like. As the glass, those having, as the main ingredient, SiO2 that is most adhesive to electrode elements such as chromium or titanium are preferred. By applying aminopropyltrimethoxy silane (APS) to this glass thin film and baking at about 120-160° C., amino groups are fixed on the surface of the glass thin film. The thickness of the electrode and the glass thin film is preferably 0.1-1 μm, respectively. It is because if the thickness of the two exceeds 1 μm, the frequency response of the piezoelectric element becomes less responsive. Furthermore, nucleotide is plated on the glass thin film of which amino groups have been coated, and incubated at 37° C. and a humidity of 90% for 1 hour in an incubator. Then, by irradiating an UV of 60 mJ/cm2 using a UV crosslinker, nucleotide is strongly fixed to the piezoelectric element.

Steps to the extraction of gene from the sample are the same as in Working Example 1. When the temperature of the gene transported in a solution to the reactor 103 is increased to about 94° C. by a temperature control mechanism 170, the gene becomes heat-denatured and become single stranded. When this single stranded gene is bound to the nucleotide fixed on the base plate 140, the oscillation frequency of the piezoelectric element changes. Thus, by measuring this change in frequency, the sequence of the gene complementary to the fixed nucleotide can be read.

When a piezoelectric element is used in the solution, a change in the solution temperature of 1° C. leads to a change in frequency of 15-30 Hz, and thus the precise control of the solution temperature is essential. In this Working Example, however, the gene-amplification reagent is not needed or the temperature cycle is not needed, and thus it has an advantage that time required for detection becomes shorter.

The foregoing has been explained with reference to Working Examples, but it should be clear to a person skilled in the art that the present invention is not limited by it in any way and various variations and modifications can be made within the spirit of the present invention and the scope of the attached claim.

1. A chip for processing of gene that is equipped with
an injection port into which a sample containing gene is delivered,
a gene extraction part into which a solution containing said sample is introduced and which has a gene-binding carrier that binds to said gene,
a washing solution-storing part that stores the washing solution to be introduced into said gene extraction part, and
a reaction part into which said gene captured in said extraction part is introduced,
wherein a fluid channel through which said washing solution is introduced from said washing solution-storing part has been connected to a region more remote from said injection port than from a region into which a solution containing said sample is introduced in said gene extraction part.

2. A chip for processing of gene that is equipped with
an injection port into which a sample containing gene is delivered,
a gene extraction part into which said sample is introduced and which has a gene-binding carrier that binds to said gene,
a washing solution-storing part that stores the washing solution to be introduced into said gene extraction part, and
a reaction part into which the gene extracted at said gene extraction part is introduced,
wherein it has been formed so that said washing solution that has been introduced from said washing solution-storing part to said gene extraction part flows into said injection port end after flowing out of said gene extraction part.

3. A chip for processing of gene that is equipped with
an injection port into which a sample containing gene is delivered,
a gene extraction part into which said sample is introduced and which has a gene-binding carrier that captures said gene,
a washing solution-storing part that stores the washing solution to be introduced into said gene extraction part, and
a reaction part into which the gene extracted at said gene extraction part is introduced,
wherein said injection port, said gene extraction part, and said washing solution-storing part have been arranged in series through a fluid channel.

4. A chip for processing of gene that is equipped with
an injection port into which a sample containing gene is delivered,
a dissolving solution-storing part that stores the dissolving solution to be introduced to said sample that has been delivered to said injection port,
a gene extraction part into which a mixture of said sample and said dissolving solution is introduced and which has a gene-binding carrier which binds to said gene,
a washing solution-storing part that stores the washing solution to be introduced into said gene extraction part,
an eluting solution-storing part that stores the eluting solution to be introduced into said gene extraction part, and
a reaction part into which said gene eluted from said eluting solution is introduced, and
that has a fluid channel which branches from between said injection port and said gene extraction part and is connected to said reaction part.

5. A chip for processing of gene that is equipped with
an injection port into which a sample containing gene is delivered,
a dissolving solution-storing part that stores the dissolving solution to be introduced into the sample that has been delivered into said injection port,
a gene extraction part into which a mixture of said sample and said dissolving solution is introduced and which has a gene-binding carrier that captures said gene,
a washing solution-storing part that stores the washing solution to be introduced into said gene extraction part, and
an eluting solution-storing part that stores the eluting solution to be introduced into said gene extraction part,
wherein either storing part of said dissolving solution-storing part, said washing solution-storing part, and said eluting solution-storing part is formed by connecting, through bending parts, a plurality of fluid channels longer in length in the longitudinal direction than in width, and the other end of said storing part has an introducing part of a fluid to be introduced when said solution stored is discharged from the storing part.

6. A chip for processing of gene according to claim 5 wherein the fluid channel constituting any of the above storing part has a maximum cross sectional area 10 times or less that of the connecting fluid channel that connects said storing part and said injection port.

7. A chip for processing of gene according to claim 5 wherein the structure of the cross section of a fluid channel constituting any of the above storing parts is such that the ratio of width and length is 10 times or less.

8. A chip for processing of gene equipped with:
the first fluid introduction part that is equipped with an injection port into which a sample containing gene is delivered, a dissolving solution-storing part that stores the dissolving solution to be introduced into the sample that has been delivered into said injection port, a gene extraction part into which a mixture of said sample and said dissolving solution is introduced and which has a gene-binding carrier that captures said gene, a washing solution-storing part that stores the washing solution to be introduced into said gene extraction part, an eluting solution-storing part that stores the eluting solution to be introduced into said gene extraction part, and a reaction part into which said gene eluted with the eluting solution is introduced, and that is located at the side more remote from said injection port than from the region in which said dissolving solution has been stored in said dissolving solution-storing part and in which a fluid is delivered to said dissolving solution-storing part when said dissolving solution is introduced into said injection port,
the first fluid discharge part that discharges the fluid in said gene extraction part out of said gene extraction part before said sample and said dissolving solution are introduced into a region more remote from said injection port than the area into which said sample and said dissolving solution are introduced in said gene extraction part,
the second fluid introduction part into which a fluid is delivered when said washing solution is introduced into said injection port at the side more remote from said gene capturing part than the area in which said washing solution has been stored in said washing solution-storing part,
the third fluid introduction part into which a fluid is delivered when said eluting solution is introduced into said injection port at the side more remote from said gene capturing part than the area in which said eluting solution of said eluting solution-storing part has been stored in said eluting solution-storing part, and
the fourth fluid introduction part into which a fluid is delivered when said solution containing said eluted gene is introduced from said gene extraction part into said reaction part.

9. An apparatus for processing of gene which is equipped with:
a chip mounting part having mounted thereon a chip for processing of gene that has an injection port into which a sample containing gene is delivered,
a gene extraction part into which a solution containing said sample is introduced and which is equipped with a gene-binding carrier that captures said gene,
a washing solution-storing part that stores the washing solution to be introduced into said gene extraction part, and
a reaction part into which said gene captured in said gene extraction part is introduced; and
a fluid introduction mechanism that introduces a fluid into said chip for processing of gene; and
a detection mechanism that detects the eluted gene,
wherein, said apparatus is controlled so that the washing solution that was introduced from said washing solution-storing part to said gene extraction part flows from said gene extraction part to said injection port.

10. An apparatus for processing of gene which is equipped with:
a chip mounting part having mounted thereon
a chip for processing of gene that has an injection port into which a sample containing gene is delivered,
a gene extraction part, formed in connection with the injection port, into which a solution containing said sample is introduced and which is equipped with a gene extraction part having a gene-binding carrier that captures said gene, and
a washing solution-storing part formed in connection with said gene extraction part, and
that is equipped with a fluid connection part of the gene extraction part downstream of said gene extraction part relative to said injection port wherein the outside and the fluid are connected, and a fluid connection part of the washing solution-storing part downstream of said washing solution-storing part relative to said gene extraction part wherein the outside and the fluid are connected;
a fluid control mechanism that introduces or aspirates a fluid into said chip for processing of gene; and
a detection mechanism that detects the gene contained in said sample,
wherein, said fluid connection part of the gene extraction part is controlled to permit the flow of a fluid between the inside of said chip and the outside of said chip, and said fluid connection part of the washing solution-storing part is controlled to limit the flow of a fluid between the inside of said chip and the outside of said chip, thereby to control to permit the introduction of a solution containing said sample from said injection port to said gene extraction part, and
said fluid connection part of the gene extraction part is controlled to limit the flow of a fluid between the inside of said chip and the outside of said chip, and said fluid connection part of the washing solution-storing part is controlled to permit the flow of a fluid between the inside of said chip and the outside of said chip, thereby to control to permit the flow of said washing solution from said washing solution-storing part through said gene extraction part to said injection port.

11. An apparatus for processing of gene according to claim 10 wherein,
said fluid connection part of the gene extraction part is controlled to aspirate and permit the flow of a fluid between the inside of said chip and the outside of said chip, and said fluid connection part of the washing solution-storing part is controlled to limit the flow of a fluid between the inside of said chip and the outside of said chip, thereby to control to permit the introduction of a solution containing said sample from said injection port to said gene extraction part.

12. An apparatus for processing of gene according to claim 10 wherein,
said fluid connection part of the gene extraction part is controlled to permit the flow of a fluid between the inside of said chip and the outside of said chip, and said fluid connection part of the washing solution-storing part is controlled to limit the flow of a fluid between the inside of said chip and the outside of said chip, thereby to control to permit the introduction of a solution containing said sample from said injection port to said gene extraction part.

13. A method of using a chip for processing of gene comprising the steps of:
cooling and freezing a chip for processing of gene having a sample injection port into which a test sample is injected, a reagent tank, in connection with said sample injection port, in which reagents have been stored, a fluid channel for extracting gene from said test sample, a reactor in which the extracted gene is detected, and a fluid channel connecting said reagent tank and the external fluid channel, and
carrying said frozen chip for processing of gene.

14. A method of using a chip for processing of gene comprising the steps of:
cooling and refrigeration a chip for processing of gene having a sample injection port into which a test sample is injected, a reagent tank, in connection with said sample injection port, in which reagents have been stored, a fluid channel for extracting gene from said test sample, a reactor in which the extracted gene is detected, and a fluid channel connecting said reagent tank and the external fluid channel, and
carrying said frozen chip for processing of gene.

15. A method of detecting gene comprising the steps of:
providing a chip for processing of gene having a sample injection port for injecting a test sample containing a gene, a reagent tank, connected to said sample injection port, in which reagents have been stored, a fluid channel for extracting gene from the test sample, a reactor for detecting the extracted gene, and a fluid channel connecting said reagent tank and the external fluid channel after the chip was once cooled, refrigerated or frozen;
bringing the provided analytical chip back to room temperature;
introducing a sample containing the gene into said sample injection port and extracting gene from said reagent with said reagent; and
detecting said gene.
